(19) United States
(12) Patent Application Publication
(10) Pub. No.: US 2017/0119264 A1
(43) Pub. Date: May 4, 2017

(54) Pulse Wave Velocity-to-Blood Pressure Calibration Prompting

(71) Applicant: Sharp Laboratories of America (SLA), Inc., Camas, WA (US)

(72) Inventor: Fredrick Hill, Portland, OR (US)

(21) Appl. No.: 14/983,348

(22) Filed: Dec. 29, 2015

Related U.S. Application Data
(63) Continuation-in-part of application No. 14/932,019, filed on Nov. 4, 2015.

(51) Int. Cl. A61B 5/021; G01L 27/00; A61B 5/022; A61B 5/00; A61B 5/0452

(52) U.S. Cl. CPC A61B 5/02125; A61B 5/7221; A61B 5/7253; A61B 5/0452; A61B 5/0059; A61B 5/02233; G01L 27/005

(57) ABSTRACT

A system and method are provided for prompting blood pressure-related calibrations. The method relies on statistical hypothesis tests over a current measurement and an accumulated set of calibration points to determine whether the benefit of calibration, in terms of calibration diversity, outweighs the cost to the patient. The algorithm uses statistical methods to predict calibration effect without actually performing the calibration, hence, reducing the calibration ‘cost’ to the patient and increasing the diversity of calibration points and thereby improving PWV-BP transform quality. The method is applicable to both manual and automatic calibration modes.

### RELATED APPLICATIONS

This application incorporates by reference an application entitled, SYSTEM AND METHOD FOR DERIVING A PULSE WAVE VELOCITY-BLOOD PRESSURE TRANSFORM, invented by Fredrick Hill, Ser. No. 14/932,019, filed Nov. 4, 2015, Attorney Docket No. SLA3572.

### BACKGROUND OF THE INVENTION

1. Field of the Invention

This invention generally relates to blood pressure measurement and, more particularly, to a system and method for determining when pulse wave velocity-to-blood pressure calibration is advantageous.

2. Description of the Related Art

In recent years, consensus has developed that a strong correlation exists between arterial pulse wave velocity (PWV) and systolic and diastolic blood pressure [1]. A PWV measurement involves a combination of simultaneous electrocardiography (ECG or EKG) and photoplethysmography (PPG) measurements. Electrocardiography is the process of recording the electrical activity of the heart over a period of time using electrodes placed on a patient's body. These electrodes detect the tiny electrical changes on the skin that arise from the heart muscle depolarizing during each heartbeat. During each heartbeat, a healthy heart has an orderly progression of depolarization that starts with pacemaker cells in the sinoatrial node, spreads out through the atrium, passes through the atrioventricular node down into the bundle of His and into the Purkinje fibers spreading down and to the left throughout the ventricles. This orderly pattern of depolarization gives rise to the characteristic ECG tracing.

Photoplethysmography is a method of measuring the perfusion of blood to the dermis and subcutaneous tissue by illuminating the tissue at the surface and observing variations of the light. With each cardiac cycle the heart pumps blood to the periphery. In recent practice, the change in blood volume caused by the pressure pulse of the cardiac cycle is detected by illuminating the skin with a light-emitting diode (LED) and measuring the amount of light either transmitted or reflected to a photodiode. The resulting waveform characterizes the relative blood volume of the tissue over time.

A blood pressure (BP) measurement based on PWV has many appealing qualities. The measurement requires no arterial compression and no recovery period. The measuring device can be small and inconspicuous and might even be worn for long periods. The measurement is very fast, producing a new blood pressure estimate on every heartbeat. As such, it is possible to estimate measurement uncertainty and reduce measurement error by methods like time averaging, uncertainty weighting, and median filters. A key challenge to the PWV-BP measurement is producing an accurate transform from PWV to BP. Characterization of the transform from PWV-to-BP can be performed by fitting a transform curve to pairs of PWV and BP samples collected from a patient. This fit effectively calibrates the transform curve to a specific patient and, as such, the (PWV, BP) pairs might be called calibration points.

Multipoint calibration can improve BP estimation significantly over single point [1]. A high quality calibration set reflects diversity in the patient state and exhibits a wide range on the measurement axes, which serves to guard against extrapolation. In currently available PWV-BP products, recalibration (if addressed at all) is triggered solely on the passage of time. Yet, passage of time does not ensure range or diversity in the calibration set. One innovative product for hospital use [11, 12] uses an integral pressure cuff to induce diversity in the calibration measurement. Recalibration is required every 4-8 hours and calibration measurements are discarded on the following calibration. This mode of operation is not appropriate for the general home-use case. Calibration is associated with a cost to the patient (e.g., time, distraction, discomfort). That cost must be considered in the calibration process, especially in the home use case where calibration is typically be performed manually and the cost may be especially high in the context of a busy patient schedule. So the challenge presented by the home use case is to balance the goal of collecting a diverse calibration set representative of recent patient state with the cost of calibration.

It would be advantageous if there existed a PWV-BP calibration prompting method able to collect a diverse calibration sets with a minimum of bother to the user, and had the capability of predicting calibration effect without actually performing the calibration.
1. Gesche, H., D. Grosskurth, G. Kuchler, A. Patzak, “Continuous blood pressure measurement by using the pulse transit time: comparison to a cuff-based method”, European Journal of Applied Physiology, DOI 10.1007/s00421-011-1983-3, May 2011.
2. SYSTEM AND METHOD FOR DERIVING A PULSE WAVE VELOCITY-BLOOD PRESSURE TRANSFORM, invented by Fredrick Hill, Ser. No. 14/932,019, filed Nov. 4, 2015, Attorney Docket No. SLA3572.
3. Teng, X., Y. Zhang, “Continuous and Noninvasive Estimation of Arterial Blood Pressure Using a Photoplethysmographic Approach”, IEEE EMBS 2003
4. Hermelling, E., “Local Pulse Wave Velocity Determination: The Arterial Distension Waveform from Foot to Crest”, PhD Thesis, 2009
5. London, E. M. et al, “Arterial Wave Reflections and Survival in End-Stage Renal Failure”, Hypertension. 2001; 38:434-438
6. McGhee, B., E. Bridges, “Monitoring Arterial Blood Pressure: What You May Not Know”, Crit Care Nurse 2002, 22:60-79
7. Zhang, G., et al, “Assessing the Challenges of a Pulse Wave Velocity Based Blood Pressure Measurement in Surgical Patients”, EMBC, 2014.
8. Zhang, G., et al, “Pulse arrival time is not an adequate surrogate for pulse transit time as a marker of blood pressure”, J Appl Physiol 111: 1681-1686, 2011
9. Caros, S., H. Loeliger, METHOD AND APPARATUS FOR A CONTINUOUS NON-INVASIVE AND NON-OBSTRUSIVE MONITORING OF BLOOD PRESSURE, U.S. Pat. No. 8,585,605 B2, Nov. 19, 2013
10. Chen, y., et al., “CONTINUOUS NON-INVASIVE BLOOD PRESSURE MONITORING METHOD AND APPARATUS”, U.S. Pat. No. 6,599,251 B2, Jul. 29, 2003
11. Banett, M., et al, “BODY-WORN SYSTEM FOR MEASURING CONTINUOUS NON-INVASIVE BLOOD PRESSURE (CNIBP)”, US Patent 2010/0160795
12. Sotera Wireless, Visi Mobile 510(k) Summary, Jun. 11, 2013.
13. Poon, C., Y. Zhang, “Cuff-less and Noninvasive Measurements of Arterial Blood Pressure by Pulse Transit Time”, IEEE EMBS 2005
14. European Heart Journal, Volume 31, Issue 19, pp. 2338-2350, June 2010.
15. DeVore, J., Statistics and Probability for Engineering and the Sciences, Eighth Edition, Brooks/Cole, 2012.

### SUMMARY OF THE INVENTION

A blood pressure (BP) measurement based on Pulse Wave Velocity (PWV) has many appealing qualities. The measurement requires no arterial compression and no recovery period. The measuring device can be small and inconspicuous and might even be worn for long periods. The measurement is very fast, producing a new blood pressure estimate on every heartbeat. As such, it is possible to estimate measurement uncertainty and reduce measurement error by methods like time averaging, uncertainty weighting, and median filters.

A PWV-BP device typically monitors the electrocardiography (ECG) and photoplethysmography (PPG) signals as the basis of its measurement. Specifically, the device measures the time interval between the ECG R-wave and the foot of the perfusion pulse [4] acquired through PPG and derives the PWV as a function of the distance from the heart to the PPG measurement site.

As disclosed herein, the short-term sample statistics of the PWV-BP measurement can be leveraged with an approach that at least ensures that 1) calibrations are reasonably spaced from previous calibrations along some meaningful dimension, 2) the spacing reflects the time from the last calibration, and 3) the calibration set evolves with the patient state. The second requirement reflects the use case and the cost of calibration. If a calibration was recently requested, the diversity payoff should be high before requesting again.

The calibration prompting algorithm rests on three statistical hypothesis tests, labeled Test 0, Test 1, and Test 2, for simplicity. These are known in inferential statistics [15] as null hypotheses.

Accordingly, a method is provided for prompting blood pressure-related calibrations. The method creates a transform of a calibration set that includes a plurality of sample pairs. Each sample pair includes a mean measurement of pulse wave velocity correlated to a measured reference blood pressure value, and each mean PWV measurement is derived from a plurality of PWV observations. After taking a current mean PWV measurement a model utility test is performed comparing an estimated slope of the transform to an estimated standard deviation of the slope (Test 1). If a null hypothesis of the model utility test is rejected, a first normalized mean difference test is performed comparing a current blood pressure estimate with a mean calibration reference blood pressure over the calibration set (Test 2). If a null hypothesis of the first normalized mean difference test is rejected, a calibration of the transform is prompted. The calibration includes augmenting the calibration set with actual BP measurements correlated to the current mean PWV measurement. If the null hypothesis of the model utility test is not rejected, a second normalized mean difference test is performed comparing the mean of the PWV over the calibration set with the current mean PWV measurement (Test 0). If the null hypothesis of the second mean difference test is rejected, a calibration of the transform is prompted.

More explicitly, performing the model utility test (Test 1) includes finding a test statistic value of t less than a first predetermined value,

$$t = \frac{{\hat{β}}_{1} - {β}_{10}}{{s}_{{\hat{β}}_{1}}}$$

where \hat{β}₁ is the estimated transform slope;

where β₁₀ is the hypothesis transform slope; and,

where s_{\hat{β}}_{₁} is the estimated standard deviation of the transform slope.

Performing the first normalized mean difference test (Test 2) includes finding the difference between the mean calibration reference blood pressure and the current blood pressure estimate normalized by a standard deviation product over the calibration set. Performing the second normalized mean difference test (Test 0) includes finding the difference between the current PWV estimate and the mean PWV over the calibration set normalized by a PWV standard deviation product.

In one aspect, performing the first normalized mean difference test (Test 2) includes adjusting the confidence threshold of the first normalized mean difference test as a function of time since the last-occurring previous calibration. Likewise, performing the second normalized mean difference test (Test 0) may also include adjusting the confidence threshold of the second normalized mean difference test as a function of time since the last-occurring previous calibration. In another aspect, performing the model utility test (Test 1), performing the first normalized mean difference test (Test 1), and performing the second normalized mean difference test (Test 0) may all, or individually include deleting the oldest elements of the calibration set as a function of time since the last-occurring previous calibration.

Additional details of the above-described method and a system for prompting blood pressure-related calibrations are presented below.

### BRIEF DESCRIPTION OF THE DRAWINGS

FIG. 1 is a schematic block diagram of a system for prompting blood pressure-related calibration.

FIG. 2 is a graph of systolic blood pressure (SBP) vs. PWV measurements depicting a calibration set (dots), estimated PWV-to-BP transform, BP estimates (squares), and BP estimate with a confidence interval.

FIG. 3 illustrates waveforms of an exemplary PWV measurement that may be acquired on every arterial pulse.

FIG. 4 shows a block diagram of the tests, the final prompt generation, and a curve fit block, which provides inputs to Test 1 and 2.

FIG. 5 is a graph depicting a continuous gating function.

FIG. 6 is a flowchart illustrating a method for prompting calibration based upon the above-described tests.

### DETAILED DESCRIPTION

### Definitions

Calibration Prompt—May be an audio tone, an active icon on a display, a voice prompt, or any indication which serves to entice the patient to calibrate the pulse wave velocity-to-blood pressure (PWV-BP) device. If a pliant patient is assumed who calibrates when (and only when) prompted, then the terms “calibrate” and “prompt” may be used interchangeably. On a worn PWV-BP device, with an integral electronically-actuated cuff measurement device, the prompt might be the cuff actuation trigger.

Feature—A characteristic of the photoplethysmography (PPG) and/or electrocardiography (ECG) signals.

Measurement—The result of an estimator (e.g., mean or median) over k observations of the PWV feature. A measurement is associated with a standard deviation over the observations and a timestamp representing the time and date of the observations. Observations typically occur once per arterial pulse. The number of observations (i.e., k) is stipulated to be greater than some minimum to improve significance of the sample statistics.

Transform Model—a linear model is typically assumed between PWV and BP or some linearizing function of the PWV and BP. The model has two parameters β₀ and β₁, representing the intercept and slope of the model equation.

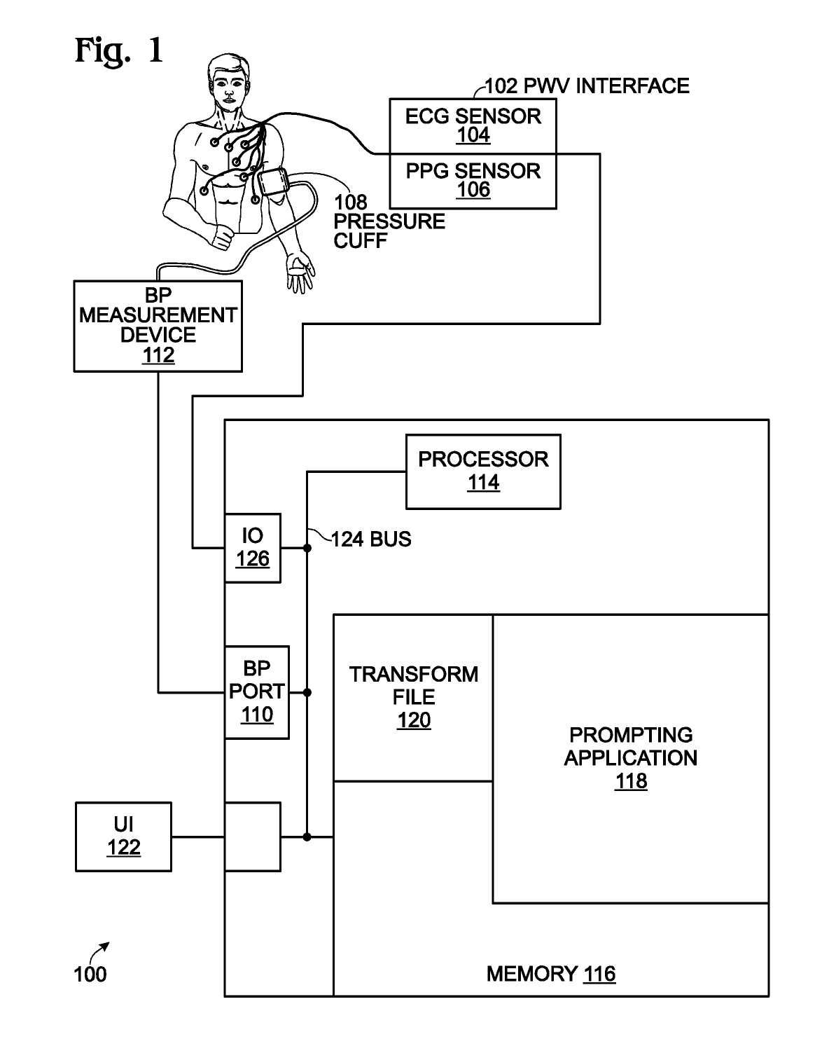

FIG. 1 is a schematic block diagram of a system for prompting blood pressure-related calibration. The system 100 comprises a PWV measurement interface 102 comprising an electrocardiogram (ECG) sensor 104 and a photoplethysmography (PPG) sensor 106 for measuring ECG and PPG signals. As noted above, the PWV feature is derived from ECG and PPG measurements. Typically, the PPG sensor 106 comprises a light emission device and a light sensing device (not shown) for detecting changes in optical transmittance of an illuminated test subject body. Typically, the ECG sensor 104 comprises at least two electrodes. The system 100 also includes a BP port 110 to accept BP measurements. As shown, a BP measurement device 112 may be connected to the BP port 110 to supply BP measurements, however taken. In one aspect, the BP measurement device 112 collects BP measurements when connected to a pressure cuff 108. As system 100 is typically used, the collection of BP measurements occurs more infrequently than PWV measurements, and in some aspects the BP port 110 is not always connected. The device further comprises a processor 114. A non-transitory memory 116 includes a prompting application 118 and a transform file 120.

For the sake of simplicity the system 100 is described in the context of a single (PWV) feature. However, as explained in more detail below, multiple features may be measured in addition to, or as an alternative to PWV. In that case, other measurement devices (not shown) may be connected to the system. The transform file 120 comprises a calibration set including a plurality of sample pairs. Each sample pair includes a mean measurement of pulse wave velocity correlated to a measured reference blood pressure values, and each mean PWV measurement is derived from a plurality of PWV observations.

The prompting application 118 is enabled as a sequence of processor instructions for accepting a current mean PWV measurement, performing a model utility test comparing the difference between an estimated slope of the transform and its hypothesis value to an estimated standard deviation of the slope (Test 1). If the null hypothesis of the model utility test is rejected, a first normalized mean difference test is performed comparing a current blood pressure estimate with a mean calibration reference blood pressure over the calibration set (Test 2). If the null hypothesis of the first normalized mean difference test is rejected, the prompting application 118 determines that the transform would benefit from calibration and issues a calibration prompt. In that case, the prompting application 118 accepts BP measurements using the cuff 108, BP measurement device 112, and BP port 110 in response to the prompt, modifies the transform by augmenting the calibration set with actual BP measurements correlated to current mean PWV measurements.

The prompting application 118 performs a second normalized mean difference test when the null hypothesis of the model utility test is not rejected, comparing the mean of the PWV over the calibration set with the current mean PWV measurement (Test 0). If the null hypothesis of the second mean difference test is rejected, a calibration of the transform is prompted.

In one aspect, the prompting application 118 performs the model utility test (Test 1) by finding a test statistic value of t less than a first predetermined value,

$$t = \frac{{\hat{β}}_{1} - {β}_{10}}{{s}_{{\hat{β}}_{1}}}$$

where \hat{β}₁ is the estimated transform slope;

where β₁₀ is the hypothesized transform slope; and,

where s_{\hat{β}}_{₁} is the estimated standard deviation of the transform slope.

The prompting application 118 calculates the estimated standard deviation of the slope (s_{\hat{β}}_{₁}) as follows:

$${S}_{{\hat{β}}_{1}} = \frac{s}{\sqrt{{S}_{xx}}} = \frac{\sqrt{\frac{SSE}{N - 2}}}{\sqrt{\sum {( {x}_{i} - \overline{x} )}^{2}}}$$

where SSE(sum of the squared errors) is defined as:

SSE=Σ(yᵢ−ŷᵢ)²;

wheres s is

$$\sqrt{\frac{SSE}{N - 2}} ;$$

where Sₓₓ is Σ(xᵢ−x̅)²;

where xᵢ is a PWV measurement in the calibration set;

where x̅ is the mean PWV over the calibration set;

where N is the number of PWV measurements within the calibration set;

where yᵢ is a reference blood pressure measurement in the calibration set; and,

where ŷᵢ is an estimated blood pressure resulting from transforming PWV measurement xᵢ.

The prompting application 118 performs the first normalized mean difference test (Test 2) by finding the difference between the mean calibration reference blood pressure and the current blood pressure estimate normalized by a standard deviation product over the calibration set. More explicitly, the prompting application 118 performs Test 2 by finding a test statistic (t) as follows:

$$t = \frac{\overline{Y} - {μ}_{0}}{\partial / \sqrt{N}}$$

where Y̅ is the blood pressure mean over the N calibration measurements;

where μ₀ is the Test 2 hypothesis mean, which is taken as the current blood pressure estimate produced by applying the transform to the current mean PWV measurement;

where ∂ is a sample standard deviation of the reference blood pressure over the calibration set.

In one aspect, the prompting application 118 performs the second normalized mean difference test (Test 0) by finding the difference between the current PWV estimate and the mean PWV over the calibration set normalized by a PWV standard deviation product. More explicitly, the prompting application 118 performs Test 0 by finding a test statistic (t) as follows:

$$t = \frac{\overline{X} - {μ}_{0}}{\partial / \sqrt{N}}$$

where X̅ is the PWV mean over the N calibration measurements;

where μ₀ is the Test 0 hypothesis mean, which is taken as the current mean PWV measurement; and,

where ∂ is estimated as a population-based standard deviation over the patient's demographic group.

In another aspect, the prompting application 118 performs the first normalized mean difference test (Test 2) by adjusting a confidence threshold of the first normalized mean difference test as a function of time since the last-occurring previous calibration. Likewise, the prompting application 118 may perform the second normalized mean difference test (Test 0) by adjusting the confidence threshold of the second normalized mean difference test as a function of time since the last-occurring previous calibration.

In one aspect, the prompting application 118 performs the model utility test (Test 1) by deleting the oldest elements of the calibration set as a function of time since the last-occurring previous calibration. In addition, the prompting application 118 may perform the first normalized mean difference test (Test 2) by deleting the oldest elements of the calibration set as a function of time since the last-occurring previous calibration. Further, the prompting application 118 may perform the second normalized mean difference test (Test 0) by deleting the oldest elements of the calibration set as a function of time since the last-occurring previous calibration.

The system 100 may also include a bus 124, input/output (IO) port 126, and user interface (UI) 122. The communication bus 124 may, for example, be a Serial Peripheral Interface (SPI), an Inter-Integrated Circuit (I²C), a Universal Asynchronous Receiver/Transmitter (UART), and/or any other suitable bus or network. Although the drawing implies that the components of the system are essentially collocated in the same device, in some aspects various components may be located outside the device, communicating with other components via a wired or wireless connection.

The memory 116 may include a main memory, a random access memory (RAM), or other dynamic storage devices. These memories may also be referred to as a computer-readable medium. Such a medium may take many forms, including but not limited to, non-volatile media, volatile media, and transmission media. Non-volatile media includes, for example, optical or magnetic disks. Volatile media includes dynamic memory. Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, or any other medium from which a computer can read. The execution of the sequences of instructions contained in a computer-readable medium (i.e. screening application 120) may cause the processor 114 to perform some of the steps of determining PWV-BP transform validity. Alternately, some of these functions may be performed in hardware (not shown). The practical implementation of such a computer system would be well known to one with skill in the art. In one aspect, the processor 114 is an ARM processor using a reduced instruction set computing (RISC) architecture.

The user interface 122 and IO port 126 may incorporate a display, a modem, an Ethernet card, or any other appropriate data communications device such as USB. The physical communication links may be optical, wired, or wireless. The user interface 122 may incorporate a keypad or a cursor control device such as a mouse, touchpad, audio speaker or alarm, touchscreen, trackball, stylus, or cursor direction keys. In one aspect of the system, the UI 122 is the component of the system delivering the calibration prompting messages to the user.

The system 100 may include a special purpose computing system, and as such, can be programmed, configured, and/or otherwise designed to comply with one or more networking protocols. According to certain embodiments, the system 100 may be designed to work with protocols of one or more layers of the Open Systems Interconnection (OSI) reference model, such as a physical layer protocol, a link layer protocol, a network layer protocol, a transport layer protocol, a session layer protocol, a presentation layer protocol, and/or an application layer protocol. For example, IO 126 may include a network device configured according to a Universal Serial Bus (USB) protocol, an Institute of Electrical and Electronics Engineers (IEEE) 1394 protocol, an Ethernet protocol, a T1 protocol, a Synchronous Optical Networking (SONET) protocol, a Synchronous Digital Hierarchy (SDH) protocol, an Integrated Services Digital Network (ISDN) protocol, an Asynchronous Transfer Mode (ATM) protocol, a Point-to-Point Protocol (PPP), a Point-to-Point Protocol over Ethernet (PPPoE), a Point-to-Point Protocol over ATM (PPPoA), a Bluetooth protocol, an IEEE 802.XX protocol, a frame relay protocol, a token ring protocol, a spanning tree protocol, and/or any other suitable protocol.

The system 100 may provide a direct connection to a remote server via a direct link to a network, such as the Internet. Connection may be provided through, for example, a local area network (such as an Ethernet network), a personal area network, a wide area network, a private network (e.g., a virtual private network), a telephone or cable network, a cellular telephone connection, a satellite data connection, or any other suitable connection.

In certain embodiments, a host adapter is configured to facilitate communication between system 100 and one or more network or storage devices via an external bus or communications channel. Examples of host adapters include, without limitation, Small Computer System Interface (SCSI) host adapters, Universal Serial Bus (USB) host adapters, IEEE 1394 host adapters, Advanced Technology Attachment (ATA), Parallel ATA (PATA), Serial ATA (SATA), and External SATA (eSATA) host adapters, Fibre Channel interface adapters, Ethernet adapters, or the like.

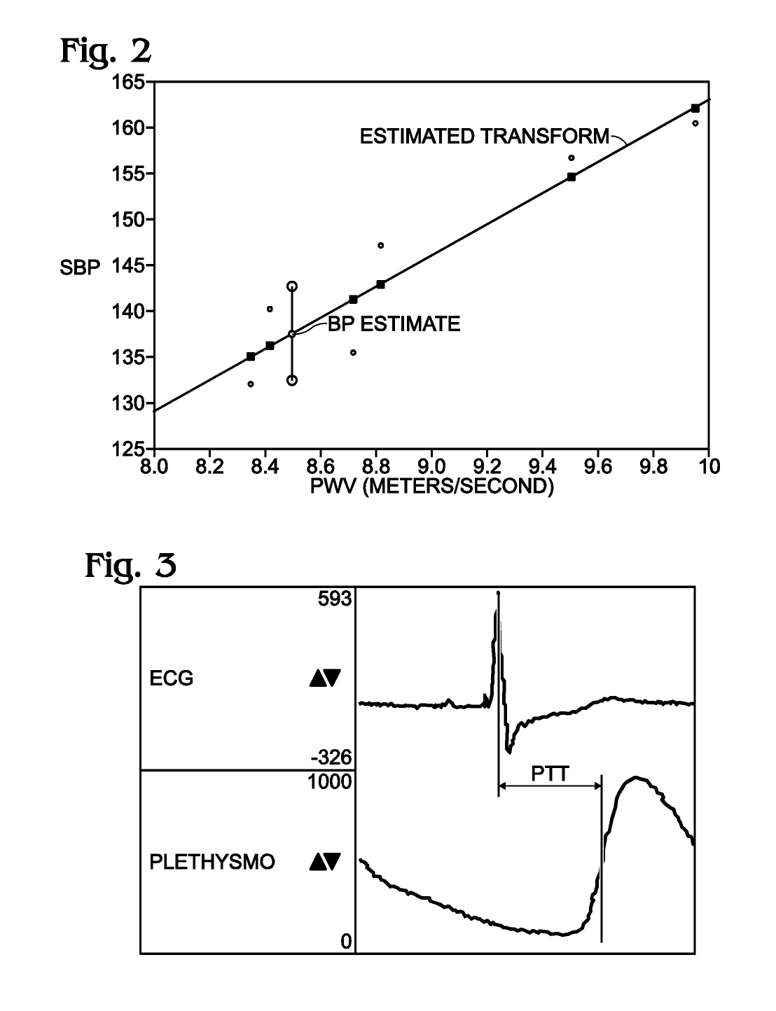

FIG. 2 is a graph of systolic blood pressure (SBP) vs. PWV measurements depicting a calibration set (dots), estimated PWV-to-BP transform, BP estimates (squares), and BP estimate with a confidence interval. Characterization of the transform from PWV to BP can be performed by fitting a transform curve to pairs of PWV and BP samples collected from a patient. This fit effectively calibrates the transform curve to a specific patient and, as such, the (PWV, BP) pairs might be called calibration points.

FIG. 3 illustrates waveforms of an exemplary PWV measurement that may be acquired on every arterial pulse. The PWV-BP system 100 typically monitors the ECG and PPG signals as the basis of its measurement. Specifically, the device measures the time interval between the ECG R-wave and the foot of the perfusion pulse [4] acquired through PPG as derives the PWV as a function of the distance traveled from the heart to the PPG measurement site.

FIG. 4 shows a block diagram of the tests, the final prompt generation, and a curve fit block, which provides inputs to Test 1 and 2. The calibration prompting algorithm rests on three statistical hypothesis tests, labeled Test 0, Test 1, and Test 2, for simplicity. These are known in inferential statistics [15] as null hypotheses.

### Test 0—Significance of PWV Difference

Test 0 is the most rudimentary of the three tests. Its null hypothesis is that the normalized difference between the mean calibration PWV and the current PWV measurement is insignificant. Hence, calibration should not be prompted. It relies only on the personal data, the current measurement, and the PWV values in the calibration history. If the calibration history is empty, the Test 0 null hypothesis is rejected (ultimately leading to a calibration prompt). Otherwise, the following test statistic is used:

$$t = \frac{\overline{X} - {μ}_{0}}{\partial / \sqrt{N}} \qquad ( 0 )$$

and has approximately standard normal distribution. X̅ is the PWV sample mean (or median) over the N calibration measurements and μ₀ is the hypothesis mean, which is taken as the current PWV measurement. (Recall that each measurement is the mean over many observations.) The parameter a (PWV standard deviation) is interpolated from published population values (e.g., [14]) based on the patient's personal data (e.g., age and gender).

The notion of p-value provides the basis for stating the test criterion. A p-value is a statistical mechanism used in hypothesis testing. It provides a frame of reference in probability for setting test limits. Specifically, the p-value is the probability of obtaining a result in the test statistic equally or more adverse to the null hypothesis than the value given by the current measurement. This probability is taken under the assumption that the test hypothesis holds. For example, assume that the current PWV is 7.0 m/s and there are two calibration points whose mean value is 6.5. The patient's age is 35, for which is derived a population-based PWV standard deviation of 2.7. This gives a t-value of

$$t = \frac{\overline{X} - {μ}_{0}}{\partial / \sqrt{N}} = \frac{6.5 - 7.0}{2.7 / \sqrt{2}} = - 0.2619 \qquad ( 1 )$$

PWV measurements greater than or equal to 7.0 or less than or equal to 6.0 would be “equally or more adverse”, yielding limits on t of ±0.2619. The probability of a t-value “equally or more adverse” is “two-sided” and (given the standard normal distribution of t) is calculated as:

p(|t|≧t₀)=2*(1−φ(t₀)) (2)

where φ(t), is the standard normal cumulative density function. For the example above:

p(|t|≧0.2619)=2*(1−φ(0.2619))=79% (3)

So, the likelihood of a PWV with greater diversity (more adverse to the null hypothesis) is quite high. Assuming a significance limit at 35% (i.e., H₀≡p(|t|≧t₀)>0.35), then the Test 0 hypothesis would “fail to reject” (because p(|t|≧0.2619)>35%). Now assume μ₀ is 8.4 m/s. Then t and the corresponding p-value are:

$$\begin{matrix}t = \frac{\overline{X} - {μ}_{0}}{\partial / \sqrt{N}} = \frac{6.5 - 8.4}{2.7 / \sqrt{2}} = - 0.9952 & ( 4 ) \\ p ( \vert t \vert > 0.9952 ) = 2 * ( 1 - ϕ ( 0.9952 ) ) = 32 \% & ( 5 )\end{matrix}$$

The null hypothesis would then be rejected. This result means that the difference in (1) is statistically significant, relative to the p-value threshold of 35%.

The algorithm uses a p-value threshold that is dependent on the time since the previous calibration and is decreasing. As such, the method ensures a statistical premium for more frequent calibrations and balances the cost of calibration (to the user) with its statistical advantage and utility in gathering a diverse calibration set. The calibration history over which the t-value is calculated is limited to a fixed time window.

This forces evolution of the calibration set to reflect recent patient state.

### Test 1 and 2—Difference Significance in Terms of Blood Pressure

Test 0 ensures meaningful calibration when few calibration points have been collected or any time when the calibration points fail to conform to the transform model. However, once the personal PWV-BP transform has been established, much of the mechanism for screening the personal transform (described in [2]) may also serve to determine calibration prompts.

From a wide perspective, Test 1 serves to establish the utility of the linear regression model in characterizing the calibration set. Test 2 assumes model utility and establishes the significance of the difference between the current BP estimate (from transformed PWV) and the mean calibration BP. Both Test 1 and Test 2 require a curve fit of the calibration parameters—a linear regression with appropriate linearization of the parameters.

Test 1 determines the ‘utility’ of the model relative to the calibration set. A small number of calibration points or a calibration set lacking diversity tends to confirm the null hypothesis that the model does not offer a useful characterization of the PWV-BP transform. Greater diversity and point count in the calibration set tends to reject the Test 1 null hypothesis and indicate that the model is useful for characterizing the relationship between PWV and BP.

Specifically, following [2] with equations renumbered:

Specifically, the Test 1 null hypothesis is confirmed when the calibration set is empty (e.g., N=0). Otherwise, Test 1 relies on the standardized variable:

$$t = \frac{{\hat{β}}_{1} - {β}_{1}}{{s}_{{\hat{β}}_{1}}} \qquad ( 6 )$$

where β₁ is the true (and unobservable) regression slope, \hat{β}₁ is the estimated slope, and s_{\hat{β}}_{₁} is the estimated standard deviation of the slope. The quantity \hat{β}₁−β₁ is an estimate residual, the difference between an estimated value and its true value. The hypothesis assumes β₁ to be a small positive value less than the smallest slope expected (e.g., 8)

The estimated standard deviation of the slope (s_{\hat{β}}_{₁}) is calculated as:

$${S}_{{\hat{β}}_{1}} = \frac{s}{\sqrt{{S}_{xx}}} = \frac{\sqrt{\frac{SSE}{N - 2}}}{\sqrt{\sum {( {x}_{i} - \overline{x} )}^{2}}} \qquad ( 7 )$$

where SSE is defined as:

SSE=Σ(yᵢ−ŷᵢ)² (8)

The numerator of (7) is the sample standard deviation of the estimate. The term (N−2) represents the degrees of freedom associated with the sum of squared errors (SSE) of the transform applied to the calibration set. The SSE is simply a squaring and summing of the residuals of the transform over the calibration set.

The Test 1 hypothesis states that

t<t_{α/2,n-2} (9)

where tₐₙ₋₂ is the value of the Student's T distribution CDF at a for n−2 degrees of freedom. The quantity t has a Student T distribution with n−2 degrees of freedom. The statistical interpretation is that over many trials, the true (unobservable) value of t lies within the stated interval with probability 1−α (e.g., 99%). Since the hypothesis is stated with a wide margin, the failure of (9) provides strong evidence that the slope of the transform is non-zero, which implies the utility of the linear model in characterizing the calibration set and implies the inferential validity of the resulting transform. Furthermore, the assumption of small positive β₁ ensures that the hypothesis is rejected only on strong evidence of a positive slope.

Test 2 depends on the rejection of the Test 1 null hypothesis. That is, Test 2 depends on model utility and, like Test 0, it is structured as a p-test. While Test 0 compares the mean calibration PWV to the current PWV measurement normalized to a population-derived PWV standard deviation, Test 2 compares the mean calibration blood pressure to the current BP estimate normalized by a product of the BP standard deviation in the calibration set. The test statistic is identical to that used for Test 0. From (0) above:

$$t = \frac{\overline{Y} - {μ}_{0}}{\partial / \sqrt{N}} \qquad ( 10 )$$

where t has approximately standard normal distribution. Y̅ is the blood pressure mean over the N calibration measurements and μ₀ is the hypothesis mean, which is taken as the current blood pressure estimate (i.e., Ŷ=\hat{β}₀+\hat{β}₁x*). The parameter ∂ is estimated as the blood pressure sample standard deviation over the calibration set.

For example, if the current systolic BP is assumed to be 122.0 mmHg, there are eight calibration points whose mean value is 120.0 and the calibration set SBPs have a standard deviation of 5.0 mmHg.

This gives a t-value of

$${t}_{0} = \frac{\overline{X} - {μ}_{0}}{\partial / \sqrt{N}} = \frac{120 - 122}{5.0 / \sqrt{8}} = - 1.1314 \qquad ( 11 )$$

The probability of a t-value “equally or more adverse” is:

p(|t|≧1.13)=2*(1−φ(1.13))=26% (12)

In this case, a reasonable likelihood of getting a “better”, more diverse, estimate may be inferred. If the current BP estimate was 128, then:

$$\begin{matrix}{t}_{0} = \frac{\overline{X} - {μ}_{0}}{\partial / \sqrt{N}} = \frac{120 - 128}{5.0 / \sqrt{8}} = - 4.5253 & ( 13 ) \\ p ( \vert t \vert \geq 4.5253 ) = 2 * ( 1 - ϕ ( 4.5253 ) ) < 0.00001 & ( 14 )\end{matrix}$$

Here, the likelihood of a more adverse estimate is 0.001%. It is quite unlikely that a subsequent measurement will be more adverse to the null hypothesis. As with Test 0, the null hypothesis for Test 2 sets a significance threshold on the p-value and assumes the p-value is greater than that threshold (hence, no prompt is needed).

While Test 2 is very similar to Test 0, Test 2 operates in the domain of blood pressure and builds both the mean and standard deviation values on actual reference measurements. Furthermore, the blood pressure estimate is based on a transform which has a verified inferential basis, courtesy of the model utility test.

Since the calibration set is composed of both systolic and diastolic measurements (two per calibration point), Test 1 and Test 2 are applied to both systolic and diastolic measurements and yield an output for each.

### Prompt Generation

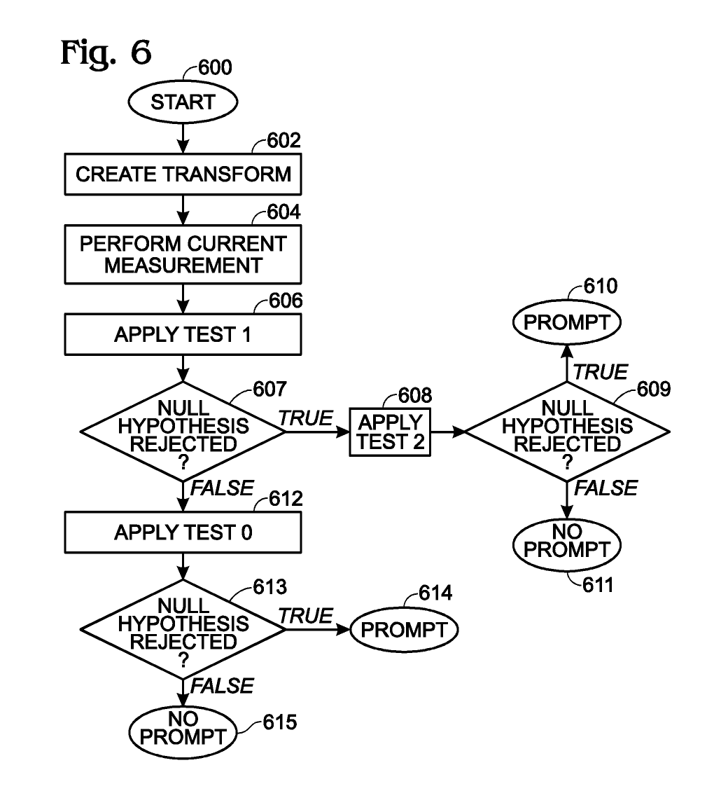

FIG. 6 is a flowchart illustrating a method for prompting calibration based upon the above-described tests. Given the hypothesis tests described above, prompt generation is straightforward. At most two hypotheses must be tested. The method begins at Step 600. A transform is derived from the calibration set in Step 602 and the current PWV measurement is collected in Step 604. Step 606 applies Test 1. If Test 1 fails to reject the null hypothesis in Step 607, then the linear model is not applicable to the calibration set. Step 612 applies Test 0. Otherwise, the linear model is established, and Step 608 applies Test 2. If Test 0 fails to reject in Step 613, no prompt is warranted (Step 615). Otherwise, Step 614 triggers a calibration prompt. If Test 2 fails to reject in Step 609, no prompt is warranted (Step 611). Otherwise, Step 610 triggers a calibration prompt.

Note that all three tests depend on the calibration history. For Test 1 and Test 2, if the history is empty, the null hypothesis for each is confirmed. For Test 0, the interpretation is different. An empty history causes the null hypothesis to reject, supporting prompt generation in cases where the calibration history is empty.

Two reference blood pressure measurements exist for each calibration measurement—systolic and diastolic. As such, the logic of FIG. 6 is applied once for systolic blood pressure and once for diastolic. A prompt is triggered to the patient if either systolic or diastolic prompts are indicated.

### Gating Function

A key goal of calibration prompting is to balance the cost of the calibration with its statistical advantage. If a calibration was recently requested, a statistical advantage (e.g., the diversity payoff) should be high before requesting again. Both Test 0 and Test 2 have thresholds that may vary to increase or decrease the level of confidence in the rejection outcome. A gating function is used to alter these thresholds as a function of time since the last calibration measurement.

For Test 0 the threshold is a p-value. For example, 35% might be used normally and a value of 10% immediately following calibration. One realization is the simple step function:

if(Δt>tThresh)pThresh=35%; else pThresh=10%;end

where Δt is the time since the last calibration, tThresh is a threshold on that time, and pThresh is the Test 0 p-value threshold below which, the null hypothesis is rejected.

For Test 2, the confidence interval threshold is chosen as a clinically insignificant range. Rather than alter that range, the interval confidence is adjusted:

if(Δt>tThresh)α=15%; else α=5%; end

These gating functions are simple step functions. If more detailed control is needed, a continuous function of time might be used. For example, the logistic function, with appropriate multipliers (m), offsets (b), and time shifts (s) might be useful:

$$g ( t ) = b + m \times ( 1 - ( \frac{{e}^{t - s}}{1 + {e}^{t - s}} ) ) \qquad ( 15 )$$

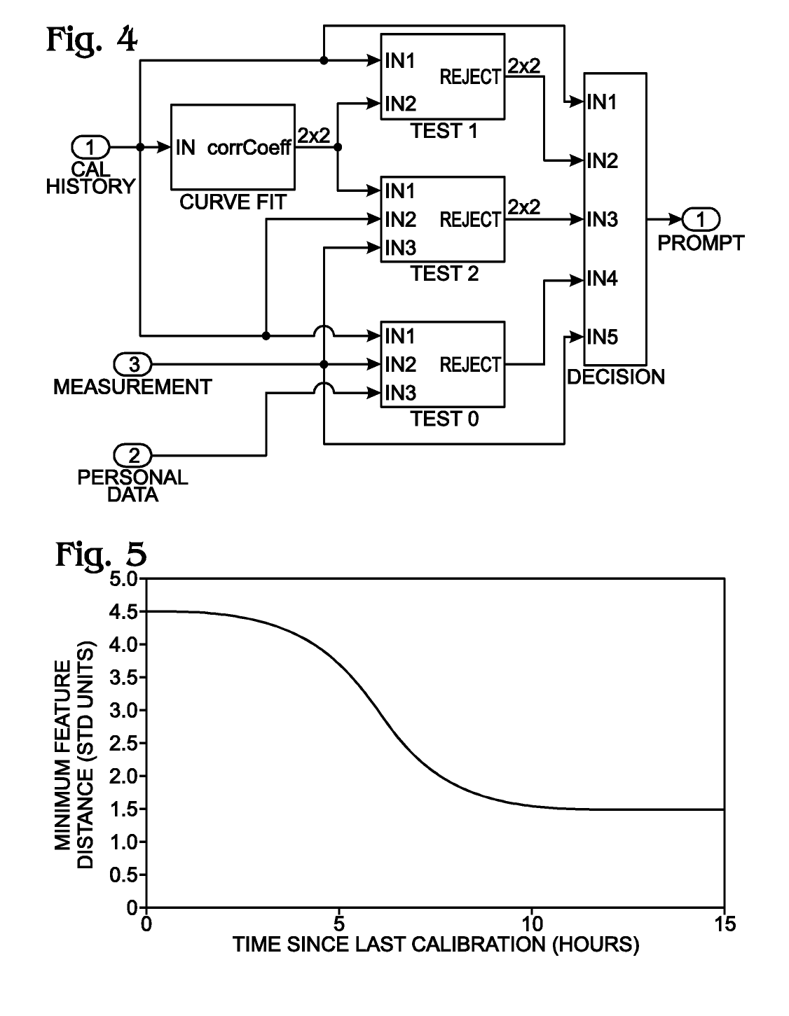

FIG. 5 is a graph depicting a continuous gating function for b=1.5, m=3, and s=6.

The algorithm is grounded in statistical methods. Its novelty derives from its capability to predict calibration effect without actually performing the calibration, hence, reducing the calibration ‘cost’ to the patient and increasing the diversity of calibration points and improving PWV-BP transform quality. The method is applicable to both manual and automatic calibration modes. It may be used in conjunction with the method described in [2] to yield a comprehensive and coherent approach to PWV-BP calibration for the home use case.

Returning to FIG. 6, as noted above, Step 602 creates a transform. The transform comprises a calibration with sample pairs including a mean measurement of pulse wave velocity correlated to a measured reference blood pressure value. Each mean measurement is derived from a plurality of feature (PWV) observations. Step 604 performs a current PWV mean measurement. Step 606 performs a model utility test comparing an estimated slope of the transform to an estimated standard deviation of the slope (Test 1). If a null hypothesis of the model utility test is rejected, Step 608 performs a first normalized mean difference test comparing a current blood pressure estimate with a mean calibration reference blood pressure over the calibration set (Test 2). If a null hypothesis of the first normalized mean difference test is rejected, Step 611 prompts a calibration of the transform, where the calibration includes augmenting the calibration sets with actual BP measurements correlated to the current mean PWV measurement.

If the null hypothesis of the model utility test is not rejected, Step 612 performs a second normalized mean difference test comparing the mean of the PWV over the calibration set with the current mean PWV measurement (Test 0). If the null hypothesis of the second mean difference test is rejected, Step 614 prompts a calibration of the transforms.

In one aspect, performing the model utility test (Test 1) in Step 606 includes finding a test statistic value of t less than a first predetermined value,

$$t = \frac{{\hat{β}}_{1} - {β}_{10}}{{s}_{{\hat{β}}_{1}}}$$

where \hat{β}₁ is the estimated transform slope;

where β₁₀ is the hypothesized transform slope; and,

where s_{\hat{β}}_{₁} is the estimated standard deviation of the transform slope.

where the estimated standard deviation of the slope (S_{\hat{β}}_{₁}) is calculated as:

$${s}_{{\hat{β}}_{1}} = \frac{s}{\sqrt{{S}_{xx}}} = \frac{\sqrt{\frac{SSE}{N - 2}}}{\sqrt{{Σ ( {x}_{i} - \overline{x} )}^{2}}}$$

where SSE(sum of the squared errors) is defined as:

SSE=Σ(yᵢ−ŷᵢ)²;

where s is

$$\sqrt{\frac{SSE}{N - 2}} ;$$

where Sₓₓ is Σ(xᵢ−x̅)²;

where x₁ is a PWV measurement in the calibration set;

where x̅ is the mean PWV over the calibration set;

where N is the number of PWV measurements within the calibration set;

where yᵢ is a reference blood pressure measurement in the calibration set; and,

where ŷᵢ is an estimated blood pressure resulting from transforming PWV measurement xᵢ.

In another aspect, performing the first normalized mean difference test (Test 2) in Step 608 includes finding the difference between the mean calibration reference blood pressure and the current blood pressure estimate normalized by a standard deviation product over the calibration set. Test 2 finds a test statistic (t) as follows:

$$t = \frac{\overline{Y} - {μ}_{0}}{\partial \text{/} \sqrt{N}}$$

where Y is the blood pressure mean over the N calibration measurements;

where μ₀ is the Test 2 hypothesis mean, which is taken as the current blood pressure estimate produced by applying the transform to the current mean PWV measurement; and,

where ∂ is a sample standard deviation of the reference blood pressure over the calibration set.

In one aspect, performing the second normalized mean difference test (Test 0) in Step 812 includes finding the difference between the current PWV estimate and the mean PWV over the calibration set normalized by a PWV standard deviation product. Test 0 finds a test statistic (t) as follows:

$$t = \frac{\overline{X} - {μ}_{0}}{\partial \text{/} \sqrt{N}}$$

where X̅ is the PWV mean over the N calibration measurements;

where μ₀ is the Test 0 hypothesis mean, which is taken as the current mean PWV measurement; and,

where ∂ is estimated as a population-based standard deviation over the patient's demographic group.

In another aspect, performing the first normalized mean difference test (Test 2) in Step 608 includes adjusting the confidence threshold of the first normalized mean difference test as a function of time since the last-occurring previous calibration. Likewise, performing the second normalized mean difference test (Test 0) in Step 612 includes adjusting the confidence threshold of the second normalized mean difference test as a function of time since the last-occurring previous calibration.

In another variation, performing the model utility test (Test 1) in Step 606 includes deleting the oldest elements of the calibration set as a function of time since the last-occurring previous calibration. In the same manner, performing the first normalized mean difference test (Test 2) in Step 608 may include deleting the oldest elements of the calibration set as a function of time since the last-occurring previous calibration. Further, performing the second normalized mean difference test (Test 0) in Step 612 may include deleting the oldest elements of the calibration set as a function of time since the last-occurring previous calibration.

A system and method have been provided for deriving BP-related calibrations. Examples of particular statistical processes have been presented to illustrate the invention. However, the invention is not limited to merely these examples. Other variations and embodiments of the invention will occur to those skilled in the art.

We claim:

1. A method for prompting blood pressure (BP)-related calibrations, the method comprising:
creating a transform comprising a calibration set including a plurality of sample pairs, where each sample pair includes a mean measurement of pulse wave velocity (PWV) correlated to a measured reference blood pressure value, where each mean PWV measurement is derived from a plurality of PWV observations;
performing a current mean PWV measurement;
performing a model utility test comparing an estimated slope of the transform to an estimated standard deviation of the slope (Test 1);
when a null hypothesis of the model utility test is rejected, performing a first normalized mean difference test comparing a current blood pressure estimate with a mean calibration reference blood pressure over the calibration set (Test 2); and,
when a null hypothesis of the first normalized mean difference test is rejected, prompting a calibration of the transform, where the calibration includes augmenting the calibration set with actual BP measurements correlated to the current mean PWV measurement.

2. The method of claim 1 further comprising:
when the null hypothesis of the model utility test is not rejected, performing a second normalized mean difference test comparing the mean of the PWV over the calibration set with the current mean PWV measurement (Test 0); and,
when the null hypothesis of the second mean difference test is rejected, prompting a calibration of the transform.

3. The method of claim 1 wherein performing the model utility test (Test 1) includes finding a test statistic value of t less than a first predetermined value,
$$t = \frac{{\hat{β}}_{1} - {β}_{10}}{{s}_{{\hat{β}}_{1}}}$$
where \hat{β}₁ is the estimated slope;
where β₁₀ is the hypothesis slope; and,
where s_{\hat{β}}_{₁} is the estimated standard deviation of the slope.

4. The method of claim 3 wherein the estimated standard deviation of the slope (S_{\hat{β}}_{₁}) is calculated as:
$${s}_{{\hat{β}}_{1}} = \frac{s}{\sqrt{{S}_{xx}}} = \frac{\sqrt{\frac{SSE}{N - 2}}}{\sqrt{{Σ ( {x}_{i} - \overline{x} )}^{2}}}$$
where SSE(sum of the squared errors) is defined as:
SSE=Σ(yᵢ−ŷᵢ)²;
where s is s
$$\sqrt{\frac{SSE}{N - 2}} ;$$
where Sₓₓ is Σ(xᵢ−ŷᵢ)²;
where xᵢ is a PWV measurement in the calibration set;
where x̅ is the mean PWV over the calibration set;
where N is the number of PWV measurements within the calibration set;
where yᵢ is a reference blood pressure measurement in the calibration set; and,
where ŷᵢ is an estimated blood pressure resulting from transforming PWV measurement xᵢ.

5. The method of claim 1 wherein performing the first normalized mean difference test (Test 2) includes finding the difference between the mean calibration reference blood pressure and the current blood pressure estimate normalized by a standard deviation product over the calibration set.

6. The method of claim 5 wherein Test 2 finds a test statistic (t) as follows:
$$t = \frac{\overline{Y} - {μ}_{0}}{\partial \text{/} \sqrt{N}}$$
where Y̅ is the blood pressure mean over the N calibration measurements;
where μ₀ is the Test 2 hypothesis mean, which is taken as the current blood pressure estimate produced by applying the transform to the current mean PWV measurement; and,
where ∂ is a sample standard deviation of the reference blood pressure over the calibration set.

7. The method of claim 2 wherein performing the second normalized mean difference test (Test 0) includes finding the difference between the current PWV estimate and the mean PWV over the calibration set normalized by a PWV standard deviation product.

8. The method of claim 7 wherein Test 0 finds a test statistic (t) as follows:
$$t = \frac{\overline{X} - {μ}_{0}}{\partial \text{/} \sqrt{N}}$$
where X̅ is the PWV mean over the N calibration measurements;
where μ₀ is the Test 0 hypothesis mean, which is taken as the current mean PWV measurement; and,
where ∂ is estimated as a population-based standard deviation over the patient's demographic group.

9. The method of claim 2 wherein performing the first normalized mean difference test (Test 2) includes adjusting a confidence threshold of the first normalized mean difference test as a function of time since a last-occurring previous calibration; and,
wherein performing the second normalized mean difference test (Test 0) includes adjusting a confidence threshold of the second normalized mean difference test as a function of time since a last-occurring previous calibration.

10. The method of claim 2 wherein performing the model utility test (Test 1) includes deleting oldest elements of the calibration set as a function of time since a last-occurring previous calibration;
wherein performing the first normalized mean difference test (Test 2) includes deleting oldest elements of the calibration set as a function of time since the last-occurring previous calibration; and,
wherein performing the second normalized mean difference test (Test 0) includes deleting oldest elements of the calibration set as a function of time since the last-occurring previous calibration.

11. A system for prompting blood pressure (BP)-related calibrations, the system comprising:
a PWV measurement interface comprising an electrocardiogram (ECG) sensor and a photoplethysmography (PPG) sensor for measuring ECG and PPG signals;
a processor;
a non-transitory memory including:
a transform file comprising a calibration set including a plurality of sample pairs, where each sample pair includes a mean measurement of pulse wave velocity (PWV) correlated to a measured reference blood pressure values, where each mean PWV measurement is derived from a plurality of PWV observations;
a prompting application enabled as a sequence of processor instructions for accepting a current mean PWV measurement, performing a model utility test comparing a difference between an estimated slope of the transform and its hypothesis value to an estimated standard deviation of the slope (Test 1), and when a null hypothesis of the model utility test is rejected, performing a first normalized mean difference test comparing a current blood pressure estimate with a mean calibration reference blood pressure over the calibration set (Test 2), and when a null hypothesis of the first normalized mean difference test is rejected, prompting a transform calibration.

12. The system of claim 11 further comprising:
a BP measurement interface comprising a BP cuff for measuring BP signals; and,
wherein the prompting application accepts BP measurements in response to prompting the transform calibration, and modifies the transform by augmenting the calibration set with actual BP measurements correlated to current mean PWV measurements.

13. The system of claim 12 wherein the prompting application performs a second normalized mean difference test when the null hypothesis of the model utility test is not rejected, comparing the mean of the PWV over the calibration set with the current mean PWV measurement (Test 0), and when the null hypothesis of the second mean difference test is rejected, prompting a calibration of the transform.

14. The system of claim 12 wherein the prompting application performs the model utility test (Test 1) by finding a test statistic value of t less than a first predetermined value,
$$t = \frac{{\hat{β}}_{1} - {β}_{10}}{{s}_{{\hat{β}}_{1}}}$$
where \hat{β}₁ is the estimated transform slope;
where β₀ is the hypothesized transform slope; and,
where s_{\hat{β}}_{₁} is the estimated standard deviation of the transform slope.

15. The system of claim 14 wherein the prompting application calculates the estimated standard deviation of the slope (S_{\hat{β}}_{₁}) as follows:
$${s}_{{\hat{β}}_{1}} = \frac{s}{\sqrt{{S}_{xx}}} = \frac{\sqrt{\frac{SSE}{N - 2}}}{\sqrt{{Σ ( {x}_{i} - \overline{x} )}^{2}}}$$
where SSE(sum of the squared errors) is defined as:
SSE=Σ(yᵢ−ŷᵢ)²;
where s is
$$\sqrt{\frac{SSE}{N - 2}} ;$$
where Sₓₓ is Σ(xᵢ−x̅)²;
where xᵢ is a PWV measurement in the calibration set;
where x̅ is the mean PWV over the calibration set;
where N is the number of PWV measurements within the calibration set;
where yᵢ is a reference blood pressure measurement in the calibration set; and,
where y̅ᵢ is an estimated blood pressure resulting from transforming PWV measurement xᵢ.

16. The system of claim 12 wherein the prompting application performs the first normalized mean difference test (Test 2) by finding the difference between the mean calibration reference blood pressure and the current blood pressure estimate normalized by a standard deviation product over the calibration set.

17. The system of claim 16 wherein the prompting application performs Test 2 by finding a test statistic (t) as follows:
$$t = \frac{\overline{Y} - {μ}_{0}}{\partial \text{/} \sqrt{N}}$$
where Y̅ is the blood pressure mean over the N calibration measurements;
where μ₀ is the Test 2 hypothesis mean, which is taken as the current blood pressure estimate produced by applying the transform to the current mean PWV measurement;
where ∂ is a sample standard deviation of the reference blood pressure over the calibration set.

18. The system of claim 13 wherein the prompting application performs the second normalized mean difference test (Test 0) by finding the difference between the current PWV estimate and the mean PWV over the calibration set normalized by a PWV standard deviation product.

19. The system of claim 18 wherein the prompting application performs Test 0 by finding a test statistic (t) as follows:
$$t = \frac{\overline{X} - {μ}_{0}}{\partial \text{/} \sqrt{N}}$$
where X̅ is the PWV mean over the N calibration measurements;
where μ₀ is the Test 0 hypothesis mean, which is taken as the current mean PWV measurement; and,
where ∂ is estimated as a population-based standard deviation over the patient's demographic group.

20. The system of claim 13 wherein the prompting application performs the first normalized mean difference test (Test 2) by adjusting a confidence threshold of the first normalized mean difference test as a function of time since a last-occurring previous calibration; and,
wherein the prompting application performs the second normalized mean difference test (Test 0) by adjusting diminishing a confidence threshold of the second normalized mean difference test as a function of time since a last-occurring previous calibration.

21. The system of claim 13 wherein the prompting application performs the model utility test (Test 1) by deleting oldest elements of the calibration set as a function of time since a last-occurring previous calibration;
wherein the prompting application performs the first normalized mean difference test (Test 2) by deleting oldest elements of the calibration set as a function of time since the last-occurring previous calibration; and,
wherein the prompting application performs the second normalized mean difference test (Test 0) by deleting oldest elements of the calibration set as a function of time since the last-occurring previous calibration.
